(19) United States
(12) Patent Application Publication
(10) Pub. No.: US 2024/0066275 A1
(43) Pub. Date: Feb. 29, 2024

(54) MULTIFUNCTIONAL EPIDURAL CATHETER BEING EQUIPPED WITH SENSOR FOR DETECTING PRESSURE APPLIED TO COMBINED BALLOON

(71) Applicant: JUVENUI Co., LTD, Seongnam-si (KR)

(72) Inventor: Hyun Hong KIM, Seoul (KR)

(73) Assignee: JUVENUI Co., LTD, Seongnam-si (KR)

(21) Appl. No.: 18/222,959

(22) Filed: Jul. 17, 2023

(30) Foreign Application Priority Data
Aug. 31, 2022 (KR) 10-2022-0110251

(51) Int. Cl. A61M 25/10; A61M 25/01

(52) U.S. Cl. CPC A61M 25/10188; A61M 25/0108; A61M 25/10182; A61M 2025/0007

(57) ABSTRACT

The present disclosure relates to a multifunctional epidural catheter being equipped with a sensor for detecting a pressure applied to a combined balloon. And, most particularly, provided herein is a multifunctional epidural catheter being equipped with a sensor for detecting a pressure applied to a combined balloon, wherein the catheter comprises a main body, and a catheter insertion part being connected to the main body and inserted in a patient's body, wherein the catheter has a combined balloon attached to one side of the catheter insertion part that is inflated when the catheter is inserted in the patient's body so as to expand an empty space within the patient's body, the multifunctional epidural catheter comprising a syringe connection part for a balloon formed on one side of the main body to protrude outside and having a syringe for the balloon connected thereto so as to supply fluid into the balloon, a fluid transfer part being formed inside the main body, having one end connected to the syringe connection part for a balloon, and transferring the fluid supplied from the syringe to be supplied to the balloon, a pressure sensor being formed inside the main body, contacting the fluid transfer part, and measuring a pressure of the fluid according to an expansion or contraction of the balloon, a display unit being formed on an external side of the main body, receiving a measurement value detected by the pressure sensor, and numerically displaying the received value, and an alarm unit generating a visual or auditory alarm when the pressure measured by the pressure sensor reaches a threshold value.

### CROSS-REFERENCE TO RELATED APPLICATIONS

This application claims priority to Korean Patent Application 10-2022-0110251, filed on Aug. 31, 2022, in the Korean Intellectual Property Office, the disclosure of which is incorporated herein by reference in its entirety.

### FIELD OF THE DISCLOSURE

The present disclosure relates to a multifunctional epidural catheter being equipped with a sensor for detecting a pressure applied to a combined balloon. And, most particularly, the present disclosure provides a catheter that can prevent a balloon from being broken or prevent an epidural space from being damaged due to an excessive amount of pressure that is applied to the epidural space by the balloon, by sensing the pressure applied to the balloon, which is positioned in an epidural space by a catheter that is inserted inside a patient's body, by a sensor that is equipped inside of the catheter.

### BACKGROUND OF THE DISCLOSURE

Spinal canal stenosis is a condition that occurs in 90% of the elderly population. In modern medicine, treatments for curing such condition are divided into non-surgical treatments and surgical treatments. Having higher risk of side effects, surgical treatments are preferred to be used as a final-state treatment method. Non-surgical treatments typically include medication treatment, balloon treatment, thermal treatment, endoscopic treatment, pulsed radio-frequency (RF) treatment, and so on. Although the treatment effects are different in each treatment types, since there is no existing equipment capable of performing multiple treatments simultaneously, each treatment is performed separately as needed.

This results in an inconvenience of having to use a separate device for each treatment. This also results in an increase in the treatment time and an increase in the degree of fatigue experienced by the medical staff conducting the treatment procedure. Therefore, an integration of multiple treatment devices into a single equipment is being requested.

Meanwhile, a balloon that is used in the balloon treatment is a treatment instrument that is used for treating lower back pains, which are caused by adhesion of an epidural space and spinal canal stenosis (or narrowing of the spinal canal), the object of which is to loosen adhesion and relieving stenosis without damaging the nerves.

The effects of the balloon treatment have already been proven in numerous medical journals. However, there have been differences in the effects including duration period, level of cure, and so on, depending upon the condition of a patient who has undergone such treatment.

This treatment should be applied by adjusting the pressure of the balloon according to the symptoms of the patient. Generally, although movements of a balloon within an epidural space is observed in real-time through a C-ARM, the C-ARM is only capable of producing tomographic images and has poor capability of drawing a distinction in the size of the balloon or the force being applied to the balloon. The medical staff conducting the treatment is only capable of conclusively distinguishing the amount of fluid (in cc) that has been injected in order to inflate the balloon. For such reason, the amount of fluid that has demonstrated the best clinical effects is being used as a model of the treatment method.

However, a problem may occur when the amount of injected fluid is used as a reference standard. This is because the shape or size of the epidural space may vary in each person due to various reasons, such as innate conditions or level of progression for the symptoms. As described above, if a standard amount of fluid is injected into a balloon that is positioned inside an epidural space, which has a different shape and size for each person, the pressure that is applied in the epidural space due to the injected amount of fluid, i.e., the inflated balloon, may vary for each case. And, if the size of the epidural space is smaller than average or has an unusual shape, there lies a problem of pain being caused or the epidural space being damaged due to the applied pressure.

For example, in an existing research, various pressure levels of 0, 50, 100, 200 mmHg were applied to the lumbar dura mater of a pig during a period of 2 to 4 hours, and changes in the nerve functions were observed. When a pressure of 0 to 50 mmHg was applied, no significant loss in the nerve condition was shown. And, when a pressure of 100 to 200 mmHg was applied, reports show that critical damage in the nerve condition was observed. According to this research, it was apparent that severe nerve damage may occur when a pressure equivalent to a systolic arterial pressure or higher is applied.

Therefore, instead of setting the amount of injected fluid as the standard level for inflating a balloon, it is appropriate to set the pressure level as the standard reference. And, accordingly, a means for accurately measuring the pressure applied to the balloon is needed.

### SUMMARY OF THE DISCLOSURE

### Technical Objects

The present disclosure has been devised to resolve the above-described technical problems of the related art. An object of the present disclosure is to allow an appropriate level of pressure to be applied to a balloon, which is positioned inside an epidural space, by determining whether or not to inject additional fluid after sensing the pressure level that is being applied to the balloon, instead of an injected amount fluid, when injecting fluid into a balloon that has reached the inside of an epidural space.

Additionally, another object of the present disclosure is to allow the pressure that is being applied by the fluid to be accurately measured, by configuring a fluid transfer path so that the fluid can to concentrated to the sensor.

Furthermore, yet another object of the present disclosure is to integrate various functions into a single catheter while relieving the burden of the patient, when inserting the catheter, by reducing the thickness of the catheter insertion part. The catheter according to the present disclosure is configured to perform medication treatment, balloon treatment, pulsed radio-frequency (RF) treatment by using a single equipment.

### Technical Solutions

In order to achieve the above-described technical objects of the present disclosure, provided herein is a multifunctional epidural catheter being equipped with a sensor for detecting a pressure applied to a combined balloon, wherein the catheter comprises a main body, and a catheter insertion part being connected to the main body and inserted in a patient's body, wherein the catheter has a combined balloon attached to one side of the catheter insertion part that is inflated when the catheter is inserted in the patient's body so as to expand an empty space within the patient's body, the multifunctional epidural catheter comprising a syringe connection part for a balloon formed on one side of the main body to protrude outside and having a syringe for the balloon connected thereto so as to supply fluid into the balloon, a fluid transfer part being formed inside the main body, having one end connected to the syringe connection part for a balloon, and transferring the fluid supplied from the syringe to be supplied to the balloon, a pressure sensor being formed inside the main body, contacting the fluid transfer part, and measuring a pressure of the fluid according to an expansion or contraction of the balloon, a display unit being formed on an external side of the main body, receiving a measurement value detected by the pressure sensor, and numerically displaying the received value, and an alarm unit generating a visual or auditory alarm when the pressure measured by the pressure sensor reaches a threshold value.

It is preferable that the fluid transfer unit extends from the syringe connection part for the combined balloon so as to contact the pressure sensor by turning upward or downward along a direction of the pressure sensor that is fixed to an upper part or lower part of the fluid transfer unit, and progresses towards the catheter insertion part.

It is preferable that a part of the fluid transfer unit contacting the pressure sensor is the fluid transfer unit contacting an entire length of a pressure sensor surface.

It is preferable that the catheter comprises a radio frequency (RF) electrode part being formed on an end of the catheter insertion part, a wire being connected to the RF electrode part, a power supply part being connected to the wire so as to supply power to the wire, and a thermocouple being configured to measure a temperature detected by a pulsed radio frequency (RF) generated from the RF electrode part. Herein, preferably, the thermocouple comprises a conductive coating layer being conductively coated by a diamond like carbon (DLC).

It is preferable that the conductive coating layer is within a range of 0.03 to 0.1 mm.

It is preferable that the RF electrode unit is connected to an end of the catheter insertion part, and wherein the RF electrode is used for a direction change of the catheter insertion part along with the wire.

### BRIEF DESCRIPTION OF THE DRAWINGS

FIG. 1 is a perspective diagram shown an overall catheter according to an embodiment of the present disclosure.

FIGS. 2A and 2B are diagrams showing a cross-sectional view of a catheter insertion part that is a component of the catheter according to an embodiment of the present disclosure.

FIG. 3 is a diagram showing a thermocouple.

FIG. 4 is a photograph showing various spinal structures of a human body including an epidural space.

FIG. 5 is an internal cross-sectional diagram of a main body of the catheter, wherein a pressure sensor according to an embodiment of the present disclosure is embedded in the main body, and wherein the catheter shows a transfer path of a fluid transfer part.

FIGS. 6A and 6B are mimetic diagrams showing a pressure level of a fluid, which is numerically indicated on the main body of the catheter according to an embodiment of the present disclosure.

### DETAILED DESCRIPTION OF THE PREFERRED EMBODIMENTS

Hereinafter, the present disclosure will be described in more detail based on the appended drawings and the preferred embodiments of the present disclosure.

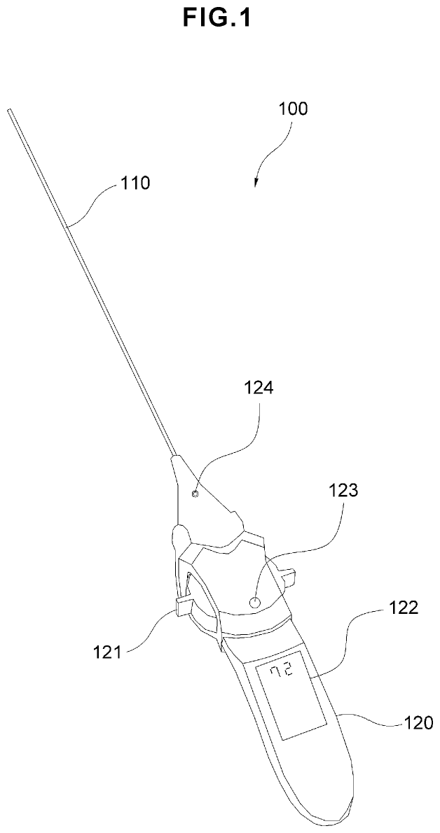

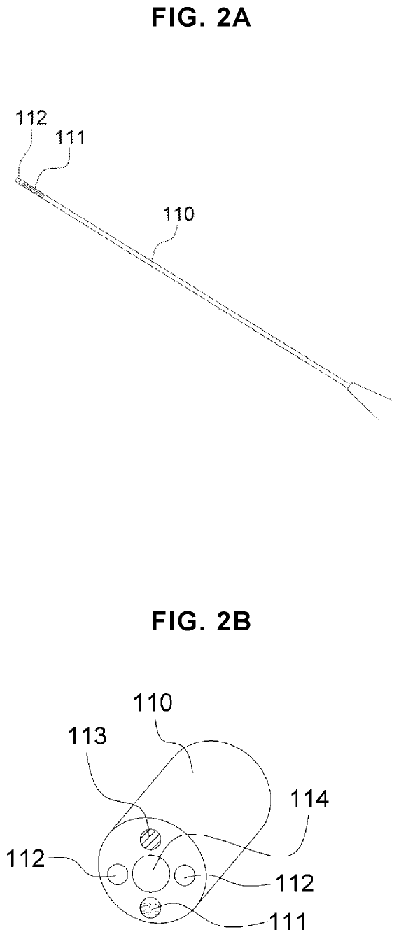

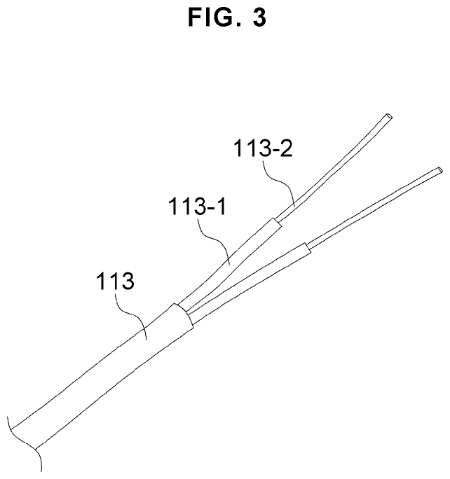

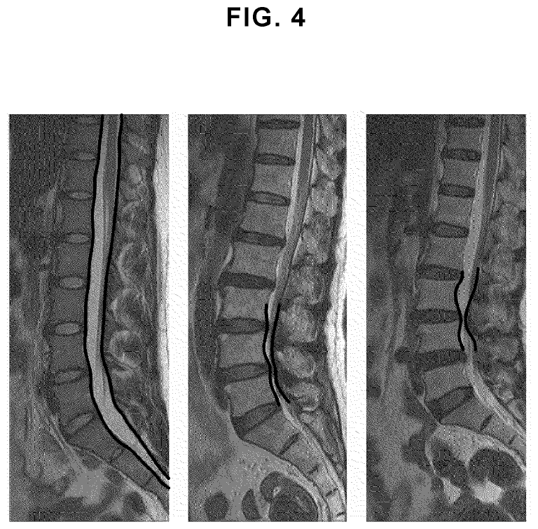

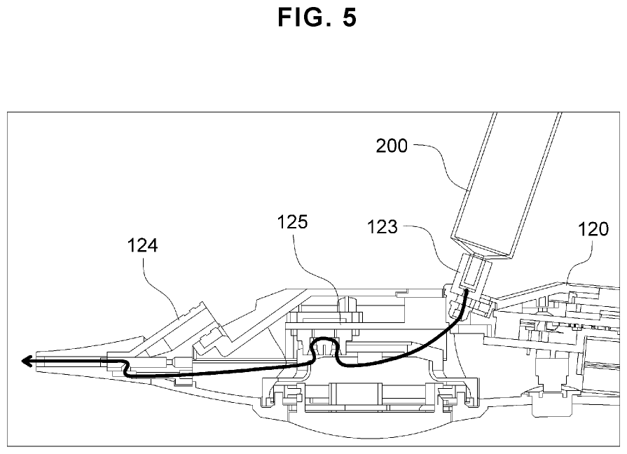

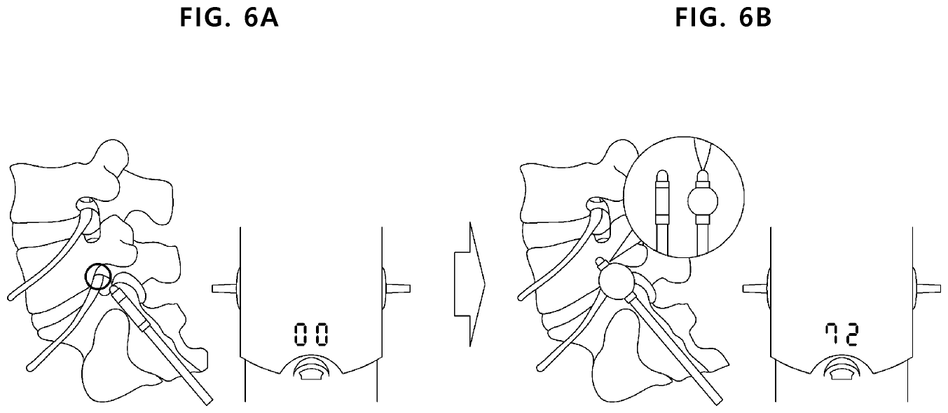

FIG. 1 is a perspective diagram shown an overall catheter according to an embodiment of the present disclosure, FIGS. 2A and 2B are diagrams showing a cross-sectional view of a catheter insertion part that is a component of the catheter according to an embodiment of the present disclosure, FIG. 3 is a diagram showing a thermocouple, FIG. 4 is a photograph showing various spinal structures of a human body including an epidural space, FIG. 5 is an internal cross-sectional diagram of a main body of the catheter, wherein a pressure sensor according to an embodiment of the present disclosure is embedded in the main body, and wherein the catheter shows a transfer path of a fluid transfer part, and FIGS. 6A and 6B are mimetic diagrams showing a pressure level of a fluid, which is numerically indicated on the main body of the catheter according to an embodiment of the present disclosure.

The essence of a non-surgical treatment is to conduct the treatment without harming (or damaging) the human (or patient's) body as much as possible. For this, the thickness of the insertion part should be maintained at its minimum limit. Herein, the corresponding device (or product) has been equipped with additional functions while maintaining the thickness of the insertion part to a same state (2.3 mm) as the conventional device (or product).

In the present disclosure, the thickness of the insertion part is set to be equal to 2.3 mm, and the insertion part has been designed to have the above-described structure in order to implement the following functions.
1. Dualization of the wire function: simultaneously implementing a function of controlling a direction of the catheter insertion part 110 and a function of supplying electric current to an electrode with its metallic property

More specifically, by using the wire for a purpose of adjusting the direction of the catheter insertion part 110 and supplying electric current to an electrode, simultaneously, a volume that is occupied by the catheter insertion part 110 may be more reduced as compared to a case where individual catheters are used for each function. The wire has been connected to the electrode so that pulsed RF treatment and lesion detection (stimulator) can be conducted.
2. Conducting balloon treatment using a side hole and conducting medication treatment using a central hole

Based on its cross-section, the catheter insertion part 110 is divided into a central part and a surrounding part (or peripheral part). By allowing medication to be injected through the central part and by allowing a balloon to be applied and function through the surrounding part, the catheter insertion part 110 is designed to be efficiently used.

As shown in FIGS. 2A and 2B, the catheter insertion part of the present disclosure is configured of a balloon (withdrawal part) 111, a wire (withdrawal part) 112, a thermocouple (withdrawal part) 113, and a medication (withdrawal part) 114, so that the catheter is capable of implementing multiple functions. Herein, the wire is designed to transfer electric current to the electrode and switching (or changing) directions of the catheter insertion part at the same time. Additionally, by reducing a thickness of the thermocouple, which will be described in more detail later on, the thickness of the catheter insertion part has been minimized.

More specifically, another structural characteristic of the present disclosure is the implementation method of a temperature sensor (thermocouple) of FIG. 3. When the thermocouple, which is used as a means to detect temperature, meets different metals each having a different conductor, the thermocouple is implemented to use a property of changing its electromotive force in accordance with the temperature. Therefore, with the exception for the temperature measuring target, insulation between metals should be maintained. The metal thickness of the thermocouple that is being produced in the market is within a range of 0.05 to 1 mm. However, since a sheath thickness is 0.2 mm or more, it is impossible to manufacture the insertion part to maintain a thickness of 2.3 mm. As the thickness becomes smaller, the cost of the insertion part increases to a considerable level. Therefore, such insertion part is not appropriate for usage as a disposable product.

Meanwhile, the electromotive force that is generated through the thermocouple has a very low pressure level of 100 mV. Therefore, in order to avoid sheathing and maintaining insulation between metals, a predetermined level of insulation resistance needs to be satisfied.

According to the present disclosure, in the thermocouple of FIG. 3, a diamond like carbon (DLC) has been used for coating a surface of a conductor in order to provide an insulating function. A thickness of a conductive coating is within a range of 0.03 to 0.1 mm, which shows a very high insulating capability as compared to its thickness. By using this method, inserting a thermocouple inside a 0.3 mm tube has become possible.

FIG. 5 shows a fluid transfer part along with a pressure sensor inside the main body 120 of the catheter 100. This is a pressure measurement structure using Pascal's principle of equal pressure being applied to an enclosed space. In order to describe a direction of application for the pressure sensor on the fluid, although the fluid transfer part (not shown) is not illustrated in detail in the drawing, the fluid transfer part is formed along the fluid transfer path, which is indicated with a red line, and is formed as a tube having a thin thickness that is almost the same as the thickness of the balloon 111. The fluid transfer part (not shown) should be formed as a thin tube in order to accurately deliver the pressure of the fluid to the pressure sensor. In FIG. 5, the pressure sensor is located above the fluid transfer part, and a portion of the fluid transfer part extends from a syringe connection part for the balloon to the inside of the main body 120 and then turns its direction upward towards the pressure sensor so as to contact the pressure sensor. Then, after passing the pressure sensor, the portion of the fluid transfer part progresses towards the catheter insertion part 110. By having the fluid transfer part turn and extend towards the pressure sensor and having the fluid transfer part preferably contact the entire length direction of the pressure sensor, the fluid transfer part may measure the pressure more accurately than a case where the fluid transfer part is extended horizontally so as to contact the pressure sensor.

When the fluid is transferred (or moves) along the fluid transfer part and, then, the fluid has a predetermined speed or higher at a portion where a change of direction occurs (a turning part), the speed of the fluid is reduced due to the friction of the tube. When excessive pressure is instantaneously generated from the syringe connection part for the balloon due to unskilled maneuvering of the device, the fluid may act as a buffer during its transfer process, thereby preventing the balloon from breaking. As the pressure changes and the fluid collides with an inner wall of the turning part, a strong pressure is applied. Since the strong pressure is directly delivered to the pressure sensor, before the actual pressure being applied to the balloon reaches a threshold value, the pressure sensor already anticipates that the threshold value of the pressure is to be applied. Thus, the breaking of the balloon or damage of the empty space within the body (e.g., epidural space), which is/are caused by the value of the pressure that is applied to the balloon exceeding the threshold value, may be prevented. Therefore, the catheter according to the present disclosure has the characteristics of more accurately measuring the pressure, adopting a method of turning (or changing) the transfer path of the fluid transfer part and positioning the pressure sensor 125 to an appropriate location in accordance with the fluid transfer part in order to take preemptive measures before the pressure reaches its threshold value.

The above-described pressure sensor may also be configured to be located below the fluid transfer part. In order to form the catheter 100 in a compact size, the pressure sensor may also be configured to have an exemplary size of 1 cm*1 cm, which is then fixed inside the main body 120.

As shown in FIGS. 6A and 6B, a degree of inflation of the balloon 111 or a change in pressure due to an obstacle may be numerically displayed on the display unit 122 so as to observe the displayed numbers and adjust the treatment intensity accordingly. Additionally, if excessive pressure is applied to the balloon 111 due to unskilled maneuvering of the medical staff conducting the treatment, there may occur a problem of having the balloon 111 break. Herein, by activating a visual, auditory, or sensory alarm at a pressure level where the balloon 111 may break (e.g., 700 to 800 atm), the breaking or destruction of the balloon 111 or, for example, damage in the empty space within the body (i.e., epidural space) or damage in nerve fascicles (or nerve bundle) within the epidural space may be prevented.

Meanwhile, an RF electrode part may be fixed to one end of the present disclosure, and by electrically connecting the RF electrode part with a wire in order to allow electric current to flow outside, electric current may flow through the RF electrode part. If electricity is connected to the RF electrode part, the RF electrode part may perform a function of a stimulator, which may replace a C-Arm X-ray (C-Arm), which is used by injecting contrast agents in order to determine a state or location of the empty space within the body (i.e., epidural space). That is, when using the stimulator of the present disclosure, since the position of the nerves can be determined by using low electric current, this method causes very little harm on the human body as compared to when using contrast agents. In some cases, the C-Arm may also be used together. However, this corresponds to a case where more precise location verification is needed. An, in this case, the risk of excessive usage of contrast agents or radiation exposure caused by excessive exposure to X-ray, which result from using the C-Arm for a long period of time, may be reduced.

The stimulator may be used in two different radio-frequency modes. The first radio-frequency of 50 Hz corresponds to a sensory mode. That is, this corresponds to a radio frequency band to which the nerves of the human body respond very sensitively. And, therefore, when electric current is applied to a lesion, the patient may feel minute pain, thereby enabling the lesion to be detected according to the patient's response. The second radio frequency of 2 Hz corresponds to a motor mode. That is, this corresponds to a radio frequency that causes minor changes in the patient's muscle. Accordingly, when electric current is applied to a lesion at a frequency causing minor changes in the muscle, the loosening and contraction of the patient's muscle can be verified through the naked eye.

As described above, the present disclosure is advantageous in that the patient's burden (or load) may be relieved by using the pressure sensor and the stimulator, damage in the balloon may be prevented, and damage in the empty space within the patient's body may be minimized.

According to the above-described present disclosure, an effect of allowing an appropriate level of pressure to be applied to a balloon, which is positioned inside an epidural space, by determining whether or not to inject additional fluid after sensing the pressure level that is being applied to the balloon, instead of an injected amount fluid, when injecting fluid into a balloon that has reached the inside of an epidural space, may be anticipated.

Additionally, according to the present disclosure, multiple functions may be integrated to one catheter, and, most particularly, by reducing the thickness of a thermocouple, which measures the temperature, the overall thickness of the catheter insertion part may be reduced, the effect relieving the burden on the patient's body during the insertion may be anticipated.

Although the present disclosure has been described in detail based on the preferred embodiments of the present disclosure, it will be apparent to those skilled in the art that the scope of the appended claims and their equivalents should not be interpreted within the limitation of the examples set forth in the description of the present disclosure.

What is claimed is:

1. A multifunctional epidural catheter being equipped with a sensor for detecting a pressure applied to a combined balloon, wherein the catheter comprises a main body, and a catheter insertion part being connected to the main body and inserted in a patient's body, wherein the catheter has a combined balloon attached to one side of the catheter insertion part that is inflated when the catheter is inserted in the patient's body so as to expand an empty space within the patient's body, the multifunctional epidural catheter comprising:
a syringe connection part for a balloon formed on one side of the main body to protrude outside and having a syringe for the balloon connected thereto so as to supply fluid into the balloon;
a fluid transfer part being formed inside the main body, having one end connected to the syringe connection part for a balloon, and transferring the fluid supplied from the syringe to be supplied to the balloon;
a pressure sensor being formed inside the main body, contacting the fluid transfer part, and measuring a pressure of the fluid according to an expansion or contraction of the balloon;
a display unit being formed on an external side of the main body, receiving a measurement value detected by the pressure sensor, and numerically displaying the received value; and
an alarm unit generating a visual or auditory alarm when the pressure measured by the pressure sensor reaches a threshold value.

2. The multifunctional epidural catheter of claim 1, wherein the fluid transfer unit extends from the syringe connection part for the balloon so as to contact the pressure sensor by turning upward or downward along a direction of the pressure sensor that is fixed to an upper part or lower part of the fluid transfer unit, and progresses towards the catheter insertion part.

3. The multifunctional epidural catheter of claim 2, wherein a part of the fluid transfer unit contacting the pressure sensor is the fluid transfer unit contacting an entire length of a pressure sensor surface.

4. The multifunctional epidural catheter of claim 1, wherein the catheter comprises:
a radio frequency (RF) electrode part being formed on an end of the catheter insertion part,
a wire being connected to the RF electrode part,
a power supply part being connected to the wire so as to supply power to the wire, and
a thermocouple being configured to measure a temperature detected by a pulsed radio frequency (RF) generated from the RF electrode part,
wherein the thermocouple comprises a conductive coating layer being conductively coated by a diamond like carbon (DLC).

5. The multifunctional epidural catheter of claim 4, wherein the conductive coating layer is within a range of 0.03 to 0.1 mm.

6. The multifunctional epidural catheter of claim 4, wherein the RF electrode unit is connected to an end of the catheter insertion part, and wherein the RF electrode is used for a direction change of the catheter insertion part along with the wire.
